(19) United States
(12) Patent Application Publication
(10) Pub. No.: US 2026/0253666 A1
(43) Pub. Date: Aug. 27, 2026

(54) NEXT GENERATION PRENATAL SCREENING

(71) Applicant: CONGENICA LTD., Cambridge (GB)

(72) Inventors: Benjamin Morgan Watkinson-Powell, Cambridge (GB); Nicholas Lench, Cambridge (GB); Suzanne Drury, Cambridge (GB)

(21) Appl. No.: 18/714,240

(22) PCT Filed: Nov. 29, 2022
(86) PCT No.: PCT/GB2022/053016

§ 371 Date: May 29, 2024

(30) Foreign Application Priority Data
Dec. 3, 2021 (GB) 2117510.4

(51) Int. Cl. G16B 20/20; G16B 20/50; G16B 30/10; G16B 40/00

(52) U.S. Cl. CPC G16B 20/20; G16B 20/50; G16B 30/10; G16B 40/00

(57) ABSTRACT

The present invention pertains to a method for determining DNA sequence variation in a fetus from samples of the fetus and parents using a Bayesian framework. The method comprising the following steps: receiving the samples comprise genomic sequence data covering one or more genomic regions of interest in relation to the fetus and the parents; processing the received samples at least in part based on a reference genome; computing a probability of fetal genotypes based on the samples using the Bayesian framework, where the Bayesian framework computes a posterior probability of whether a DNA sequence variant is present in the samples based on a prior probability and a likelihood function; and determining, based on the posterior probability of fetal genotypes, whether the DNA sequence variant is present in the fetus.

The present application relates to a system, apparatus and method(s) for next generation prenatal screening and diagnosis using a Bayesian framework.

### BACKGROUND

Genomics research has led to the increased importance of early detection of inherited and acquired disease-causing DNA sequence variants or gross chromosomal changes in both germline and somatic DNA. Testing for a specific mutation in fetal DNA may help direct clinicians in administering an appropriate actionable intervention (for example, management, treatment, therapy) for the baby based on the findings.

Before the advent of next generation sequencing (NGS), conventional DNA testing looked at only a single or a few mutations. Empowered by NGS, a clinician can simultaneously investigate multiple genes related to a particular disease phenotype (e.g. rare disease, inherited cancer). NGS methods can be performed on genetic material obtained from cell free DNA (cfDNA) obtained from blood samples non-invasively (i.e. without the need for chorionic villus sampling or amniocentesis) from a pregnant woman, the results of which enable the analysis of the genetic makeup of the fetal genome contained in the cell free fetal DNA (cffDNA).

Various methods designed to identify de novo mutations from cfDNA have used a series of bioinformatics filters to exclude variants, which are judged false positives, from a large initial list of candidate variants. Data resulting from these filters are processed in an ad hoc sequential manner rather than in a consolidated and rigorous statistical solution.

One such NGS method is published by Chan, K. et al. The publication describes a method for noninvasive fetal genome analysis that reveals de novo mutations, single-base parental inheritance and preferred DNA ends.

The use of sequential bioinformatics filters to identify de novo mutations is a relatively inaccurate approach that does not combine all of the available information in a unified statistical framework and so does not provide a reliable estimate of the probability that a variant is present. This may result in the more frequent calling of genetic variants that are not truly present in the sample (false positives) and/or the exclusion of variants that are in fact present (false negatives).

Another such NGS method is published by Rabinowitz, T. et al. The method described in the publication is designed to call inherited genetic variants from cfDNA samples. The method does not include the calling of de novo mutations.

The exclusion of de novo mutation calling from the existing Bayesian statistical based method means that some potential disease-causing genetic variants cannot be identified (using this method) in a clinical setting.

For these above reasons, an improved method for calling de novo mutations, more specifically DNA sequence variant, from cfDNA samples is required that can be used in a clinical setting to identify, with a high degree of accuracy and confidence, disease-causing genetic variants, or more specifically pathogenic DNA sequence variants, in a fetus during pregnancy. The non-invasive nature of the cfDNA sampling process minimizes any risk and distress associated with established invasive DNA sampling procedures such as miscarriage. The identification of pathogenic DNA sequence variants will enable clinicians to make informed decisions regarding the mother and baby's care.

The embodiments or aspects described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

In a first aspect, the present disclosure provides a method for determining DNA sequence variation in a fetus from samples of the fetus and parents using a Bayesian framework, the method comprising: receiving the samples comprise genomic sequence data covering one or more genomic regions of interest in relation to the parents; processing the received samples at least in part based on a reference genome; computing a probability of fetal genotypes based on the samples using the Bayesian framework, wherein the Bayesian framework computes a posterior probability of whether a DNA sequence variant is present in the samples based on a prior probability and a likelihood function; and determining, based on the posterior probability of fetal genotypes, whether the DNA sequence variant is present in the fetus.

In a second aspect, the disclosure provides a system for DNA sequence variant detection using a Bayesian framework, wherein the system comprising: an input module configured to receive samples comprise genomic sequence data covering one or more genomic regions of interest in relation to at least one parent; a processing module configured to pre-process received samples at least in part based on a reference genome; a Bayesian framework configured to compute a probability of fetal genotypes based on the samples, wherein the Bayesian framework is adapted to calculate a posterior probability of whether a DNA sequence variant is present in the samples based on a prior probability and a likelihood function; and an output module configured to determine, based on the posterior probability of fetal genotypes, whether the DNA sequence variant is present in a fetus.

In a third aspect, the disclosure provides a system for DNA sequence variant detection using a Bayesian framework, wherein the system comprises at least one processor configured to execute the method of the first aspect.

In a fourth aspect, the disclosure provides a device, wherein the device comprises one or more processors; one or more memory; and one or more programs, wherein said one or more programs are stored in said one or more memory and configured to be executed by said one or more processors; said one or more programs including instructions according to the method the first aspect.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software, which “describes” or defines the configuration of hardware, such as HDL (hardware description language) software, as is used for designing silicon chips, or for configuring universal programmable chips, to carry out desired functions.

Thereafter described options or optional features may be combined with any of the above aspects of the invention as appropriate and apparent to a skilled person.

### BRIEF DESCRIPTION OF THE DRAWINGS

Embodiments of the invention will be described, by way of example, with reference to the following drawings, in which:

FIG. 1 is a flow diagram illustrating an example of fetal DNA sequence variant detection using a Bayesian framework;

FIG. 2 is a schematic diagram illustrating an example of system for fetal DNA sequence variant detection based on samples of fetus and both parents;

FIG. 3 is a schematic diagram illustrating an example of the Bayesian framework applied to the genomic sequences data for detection of de novo DNA sequence variants; and

FIG. 4 is a block diagram of a computing device or apparatus suitable for implementing embodiments of the invention.

Common reference numerals are used throughout the figures to indicate similar features.

### DETAILED DESCRIPTION

Embodiments of the present invention are described below by way of example only. These examples represent the best mode of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

The present disclosure relates to the use of statistical methods such as a Bayesian statistical framework and non-invasive DNA sampling techniques to identify fetal genetic variants for clinical purposes. More specifically, the framework to call (genotype) fetal de novo DNA sequence variants from cell free DNA (cfDNA) samples. The samples are taken from pregnant mothers using non-invasive prenatal testing (NIPT) methods. The de novo DNA sequence variants can be either single nucleotide variants (SNVs), or short indels. These DNA sequence variants have not been inherited from the parents. In accordance, the use of the Bayesian statistical framework as part of the overall method is purposed to accurately identify potentially pathogenic DNA sequence variants in a fetus during pregnancy, while minimizing the risk and potential distress to the mother caused by invasive sampling. The identification of these variants can be used in a clinical setting to help ensure that the most appropriate actionable interventions with respect to one or more genomic regions of interest can be made.

In more detail, the method combines available relevant sequence data from the cfDNA sample, as well as from parental DNA samples, and uses a purpose built Bayesian statistical model/framework that is specifically designed to calculate the probability of different potential fetal genotypes being present within the fetal DNA at the relevant loci. The Bayesian statistical model calculates the prior probability that a de novo DNA sequence variant is present in the fetus using a function that calculates the expected mutation rates for SNVs or indels. The method also takes into account the fact that cfDNA is composed of a mixture of DNA that belongs to the mother and DNA which is derived from the fetus. This is achieved by calculating the fraction of the cfDNA that pertains to the fetus, for different binned DNA read lengths, and using this calculation to estimate the probability that each DNA read originates from the fetal DNA. This process therefore uses DNA read lengths, which are generally shorter on average for cell free fetal DNA (cffDNA) versus cell free maternal DNA reads, to inform the called fetal genotype. As per Bayesian methodology, the prior probabilities and DNA read evidence for different fetal genotypes are combined to produce the estimated posterior probability of a given genotype at a given locus. The most probable genotype is then used to indicate whether or not a de novo DNA sequence variant is present in the fetus.

The present disclosure of the Bayesian statistical framework and/or non-invasive DNA sampling techniques improves the identification of de novo variants in fetal DNA. Compared with previous documented approaches, the present disclosure is more rigorous and potentially more accurate, while making the best use of non-invasive prenatal testing. As a result, significantly clinical health risks are reduced, creating less distress for the mother. The identification of a pathogenic de novo DNA sequence variant in the fetus can enable the implementation of actionable interventions (e.g. management, treatment, therapy) for the benefit of the baby and the mother in both prenatal and neonatal periods. The management or the underlying strategies, for example, may include but are not limited with respect to identified diseases/genetic disorders such neurodevelopmental disorders, skeletal dysplasias, cardiac malformations that are known to be caused by a high incidence of de novo mutations. The management may lead to direct treatment, surgery or therapy and may include a dietary intervention e.g. ketogenic diet for epilepsy.

One example of an actionable intervention may be dietary management by way of limiting dietary galactose or affecting galactose metabolism. Dietary management can be an effective, actionable intervention for diseases arising from genes: GALT, GALE, GALK1, and GALM.

Another example of an actionable intervention may be the use of hematopoietic stem cell transplantation (HSCT). HSCT is effective for disorders arising from dysfunction or failure of one or more stem cell lineages, causing disorders such as severe combined immunodeficiency, thalassemia, and congenital amegakaryocytic thrombocytopenia. HSCT is also used to treat genetic abnormalities in CLCN7, TCIRG1, SNX10, and TNFRSF11A, leading to osteopetrosis. Additionally, HSCT has been used in a number of storage disorders (Krabbe disease, mucopolysaccharidosis (MPS) as a way to supply the missing enzyme.

Further examples of actionable intervention include but are not limited to enzyme replacement therapy, solid organ transplantation, use of supplements, immunoglobulin therapy, medications, vaccinations, and blood products, gene therapy or treatment, other medical procedures such as phlebotomy, colonoscopy with polyp removal, endoscopy with polyp removal, apheresis, prophylactic mastectomy, prophylactic oophorectomy, transsphenoidal surgery, thyroidectomy, colectomy, bilateral adrenalectomy, and pancreatic resection.

With respect to the present disclosure describing the underlying invention to achieve improved performance and accuracy in predicting DNA sequence variants, the input of high coverage DNA sequence data from the cfDNA sample from the mother and genomic DNA samples from both the mother and father are taken and utilized. These sequence data include coverage of the genomic region(s) of interest. The sequence data are aligned to a reference genome. The aligned sequence data are inputted to the purpose built Bayesian solution that performs the required statistical variant calling steps and the identification of de novo DNA sequence variants in addition to the improved identification of inherited variants in fetal DNA. More specifically, the improved identification and corresponding method described herein provide variant calls at loci where multiple ALT alleles can be potentially inherited from the parents as opposed to restricting to only one ALT allele like in the Rabinowitz et al.

Further, in the present disclosure, DNA sequence variants refer to the changes in the DNA sequence of a gene, resulting in a variant of such gene. Such variants may include alteration of single base units in, for example, DNA, or the deletion and insertion. The variants may be disease-causing or pathogenic DNA sequence variants. The DNA sequence variants may be inherited or de novo. Examples of the DNA sequence variants may include but are not limited to single nucleotide variants (SNVs) and (short) indels.

An inherited DNA sequence variant refers to an acquired mutation in the DNA from at least one parent. Such mutation may be acquired from both parents.

A de novo DNA sequence variant refers to a genetic alteration that is present for the first time as a result of a variant (or mutation) in a germ cell (egg or sperm) of one of the parents or a variant that arises in the fertilized egg itself during early embryogenesis. In some cases, the de novo DNA sequence variant could be absent in the zygote but is acquired sometime later.

Samples refer to blood samples, DNA samples, cell free DNA samples (cfDNA), cell free maternal DNA samples, cell free fetal DNA (cffDNA) samples, samples corresponding to or associated with DNA sequence data, or a combination thereof. One example of samples or sample may be a DNA sample such as a cfDNA sample taken from the blood of pregnant mothers. The DNA sample may be obtained using one or more non-invasive prenatal testing (NIPT) methods. Another example may be a cffDNA sample obtained from the maternal blood of the pregnant mother with respect to the fetus. Exemplary samples and DNA from such samples may be taken from both mother and father.

In another example, the samples taken may encompass high molecular weight genomic DNA from the sample taken from both the mother and father or DNA sequence data (in FASTQ format) from the cfDNA or cffDNA sample taken from the mother. The genomic or DNA sequence data may cover the genomic region(s) of interest. The genomic region(s) of interest is an area or segment of the gene associated with one or more DNA sequence variants or disease-causing or pathogenic DNA sequence variants. The genomic region or regions of interest may cover multiple genes of interest, which are potentially associated with inherited or acquired genetic disorders or pathogenic DNA sequence variants. The genomic region(s) of interest may correspond to genome-wide association study (GWAS) in associating specific genetic variations with particular diseases. The genomic region(s) of interest may be based on relevant publications or evidence.

Herein described cffDNA differentiates from cfDNA. The cfDNA generally refers to samples from the mother, encompassing both cell free DNA derived from the mother's genome as well as cell free fetal DNA (cffDNA) derived from the fetal genome; the cffDNA refers to cell free fetal DNA samples contained within the maternal blood plasma or cell free maternal DNA samples with respect to or more specifically derived from the fetus. It is further understood that samples of the fetus refer to cffDNA exclusively. The samples of the fetus do not refer to samples directly taken from the fetus.

The genomic region(s) of interest may be a subset of genes with at least one actionable intervention. The subset set of genes may be a TREATOME® currently ascribed to 619 genes corresponding to 633 disease entities. This subset of 619 genes is selected from a collection of 4,339 genes associated with phenotype-causing variants in relation to the DNA sequence variant herein described. The TREATOME content of 619 genes can be expanded to include additional genes as new actionable interventions are developed.

Further computing is required to carry out processing the genomic sequence data for input to the Bayesian framework. For example, a cluster computing environment may be deployed carrying out the steps of the bioinformatics pipeline. Alignment of the genomic sequence data to a reference genome is performed independent of the pipeline or the Bayesian framework. The genomic sequence data may be aligned before inputting the resultant BAM files into a purpose-built software solution as described herein that carries out the required statistical variant calling within the Bayesian framework.

The reference genome refers to any digital nucleic acid sequence database assembled by a skilled person as a representative example of the set of genes in one idealized individual organism of a species. The reference genome is deployed by one or more alignment software for aligning the genomic sequence data.

Fetal genotypes refer to the genotype of a fetus, including a single or collection of genes. Fetal genotypes may be inherited alleles or alleles associated with de novo DNA sequence variants in a particular gene. The clinical presentation of the genotype (i.e. phenotype) is a result of the transcription and translation (expression) of the gene. An abnormal fetal phenotype may be predicted by variant calling and the identification of pathogenic DNA sequence variants in the fetus. The most probable fetal genotype generated by the Bayesian framework may be indicative or highly probable of whether or not a de novo DNA sequence variant or inherited DNA sequence variant is present in the fetus. An example of the fetal genotype may correspond to a pathogenic DNA sequence variant.

The different lengths in the genomic sequence data may be used for determining a fraction of fetal-derived DNA in the genomic sequence data. The fraction of fetal-derived DNA is computed separately for different binned fetal fragment lengths. The result of which may serve as an approximation to using a statistical distribution such that the statistical distribution representative of the genomic sequence data may be determined. The statistical distribution comprises fractions of DNA orientated from the fetus or fetal-derived and DNA orientated from the mother or maternal-derived, making up for the fetal-derived and maternal-derived fractions. The Bayesian framework may use the fetal-derived fractions to compute a probability of fetal genotypes given the genomic sequence data at each region of interest.

The range of inherited alleles may be any other allele(s) that are found at that locus or referred to as the ALT allele(s). These inherited alleles correspond to the genotypes of the parents. The probability that the fetus has inherited the ALT allele(s) may be computed using the Bayesian framework.

Further, it is understood that fragment genotypes that have not been called in either parent, and are not candidate de novo DNA sequence variants, may still arise due to sequencing error. The probability of this sequencing error occurring is taken into account as part of the likelihood function in the described Bayesian framework. This applies even if the fragment genotype could also have arisen through inheritance or de novo DNA sequence variants.

In relation to the above, the Bayesian framework refers to using the Bayes formulation or theorem, where P(A|B)=P(B|A)×P(A)/P(B) is used to compute the probability event A occurring. In this case, the probability is computed in relation to fetal genotypes given the genomic sequence data or P(G|data). Chain rule or general product rule may be applicable in the computation, which describes a probability distribution in terms of conditional probabilities. At each region of interest, the Bayesian framework is applied. For each possible fetal genotype,

$$P ( G \vert data ) = \frac{P ( data \vert G ) P ( G )}{{\sum }_{i = 1}^{n} P ( data \vert {G}_{i} ) P ( {G}_{i} )}$$

where G is the fetal genotype, and Gᵢ is the i^{th} possible fetal genotype of n possibilities. The prior probability of each genotype is denoted as P(G) and is calculated based on the expected mutation rate for de novo mutations or DNA sequence variants, and on Mendelian inheritance probabilities for inherited DNA sequence variants. The data are the reads that cover a site, and P(data|G) is the likelihood function, which is a product of the likelihood of each read-observation:

$$\begin{matrix}P ( data \vert G ) = {\prod }_{j = 1}^{m} P ( {r}_{j} \vert G , {G}_{M} , f ) \\ = {\prod }_{j = 1}^{m} ( P ( {r}_{j} \vert fet ) P ( fet ) + P ( {r}_{j} \vert mat ) P ( mat ) )\end{matrix} .$$

The likelihood of a read rⱼ depends on the tested fetal genotype G and is calculated using the maternal genotype G_{M} and the fetal fraction f. P(rⱼ|fet) and P(rⱼ|mat) are the probabilities of a read-observation that supports a certain allele, given that the read is fetal and maternal, respectively. These probabilities depend on the tested fetal genotype G and maternal genotype G_{M} respectively, along with the observed allele rⱼ. P(fet) and P(mat) are the probabilities of observing a fetal or maternal read based only on the fetal fraction, regardless of the allele that it supports. The relevant fetal fraction for a given genetic locus may depend upon the lengths of the DNA fragments that cover the locus in question.

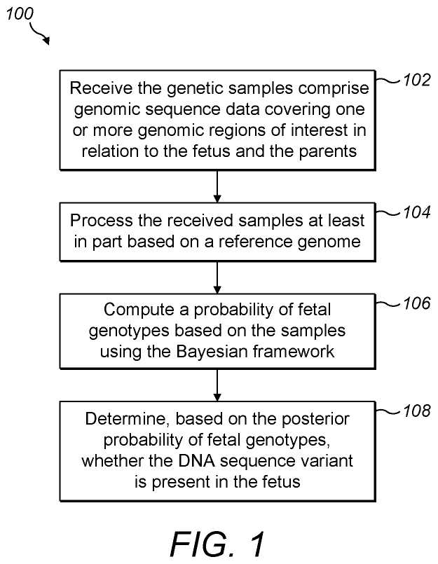

FIG. 1 is a flow diagram 100 illustrating an example method of fetal variant detection using a Bayesian framework. The presence of fetal variants(s), or specifically pathogenic DNA sequence variant(s), is determined using DNA samples obtained from the fetus (or cffDNA) and both parents. The method deploys a Bayesian framework to compute the probability of whether a pathogenic DNA sequence variant is present or not present.

In step 102, the DNA samples are received as method input, where the DNA samples comprise genomic sequence data covering one or more genomic regions of interest in relation to the fetus and the parents. For example, the samples may comprise DNA taken from a pregnant mother of the fetus and fetal cfDNA (cffDNA) samples taken from the maternal blood of the pregnant mother. The samples may also comprise DNA taken from the father of the fetus.

In step 104, the received samples are processed at least in part based on a reference genome. In the processing, the genomic sequence data may be aligned to the reference genome representative of a haploid mosaic of different DNA sequences from the donors. The sequence alignment to the reference genome may be handled using one or more suitable sequence alignment solutions. Based on the alignment results, potential variants in the DNA samples may be detected and identified. The step to process the samples may be independent of the Bayesian framework or lead to input thereof.

Further, a proportional distribution may be computed for or with respect to the samples, where the samples are representative of a fetal-derived fraction and a maternal-derived fraction of the samples. The computation of the proportional distribution may be based on lengths of DNA fragments associated with each fraction in relation to the genomic sequence data. From binned fetal fragment lengths, for example, the fraction of fetal-derived DNA may be computed. The result of the computation may serve to approximate the proportional distribution. The proportional distribution is thereby representative of the genomic sequence data underlying the fetal-derived fraction and a maternal-derived fraction of the samples. The computed proportional distribution may be applied in relation to the Bayesian framework to determine whether the pathogenic DNA sequence variant is present in the fetus.

In step 106, a probability of fetal genotypes is computed based on the samples using the Bayesian framework. The Bayesian framework may account for the different lengths in the genomic sequence data and the proportion of fetal-derived DNA fraction in the genomic sequence data. The probability computed using the Bayesian framework may be calculated with respect to testing the presence of potential fetal genotypes within a fetal-derived fraction of the samples.

More specifically, the Bayesian framework computes a posterior probability of whether a mutation, or specifically a pathogenic DNA sequence variant, is present in the samples based on a prior probability and a likelihood function. The prior probability may be calculated based on an expected mutation rate for a de novo DNA sequence variant or the probability of Mendelian inheritance for an inherited DNA sequence variant.

With respect to the likelihood function, a variable error rate may be provided and applied with respect to likelihood calculations of a de novo pathogenic DNA sequence variant, an inherited DNA sequence variant, and/or a range of inherited alleles based on parental genotypes. An error model may provide the error rate. The error model may also be configured to use locus-specific error rates based on the genomic sequence data in the likelihood calculations, wherein the likelihood calculations compute P (genetic sequence data|fetal genotypes).

The DNA sequence variants present in the DNA sample may comprise inherited and de novo variants, for example, such as single-nucleotide polymorphisms (SNVs) and short indels. Whether a DNA sequence variant is a de novo or an inherited is determined in relation to the Bayesian framework for calculating the prior probability.

In step 108, based on the posterior probability of fetal genotypes, a determination is made with respect to whether the DNA sequence variant is present in the fetus. This determination is provided as a result computed using the Bayesian framework.

Further, the probability of fetal genotypes may be recalibrated using one or more machine learning (ML) models to determine whether the DNA sequence variant is a false positive or a false negative. Said one or more ML models may be trained using data associated with de novo and inherited DNA sequence variants.

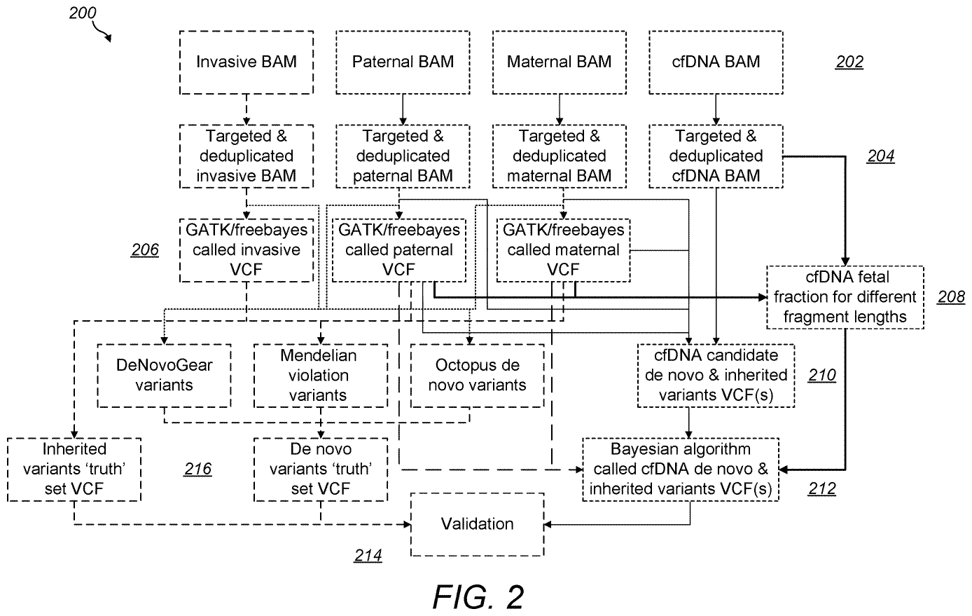

FIG. 2 is a schematic diagram 200 illustrating an example of a system for fetal DNA sequence variant detection based on samples of the fetus and both parents. The system determines fetal DNA sequence variant at least in part or principally using a Bayesian framework, wherein the system comprises one or more modules configured to process the samples in the form of genomic sequence data inputted to the system. The system outputs a determination of whether the DNA sequence variant is present in a fetus based on the computed results of the Bayesian framework.

More specifically, the system comprises an input module configured to receive samples comprising genomic sequence data that may cover one or more genomic regions of interest in relation to the parent; a processing module configured to pre-process received samples at least in part based on a reference genome; a Bayesian framework configured to compute a probability of fetal genotypes based on the samples, wherein the Bayesian framework is adapted to calculate a posterior probability of whether a DNA sequence variant is present in the samples based on a prior probability and a likelihood function; and an output module configured to determine, based on the posterior probability of fetal genotypes, whether the DNA sequence variant is present in a fetus.

In relation to the figure, the input module receives input with high coverage DNA sequence data (in FASTQ format) from the cfDNA sample taken from the mother and DNA samples taken from both the mother and father. This sequence data will include coverage of the genomic region(s) of interest. A suitable computing environment with appropriate hardware configuration is setup to process input received by the input module. The different colored lines in the figure are to distinguish between shared sets of input files with respect to the system. For example, the parental BAMs/VCFs may feed into generating or providing candidate de novo VCF.

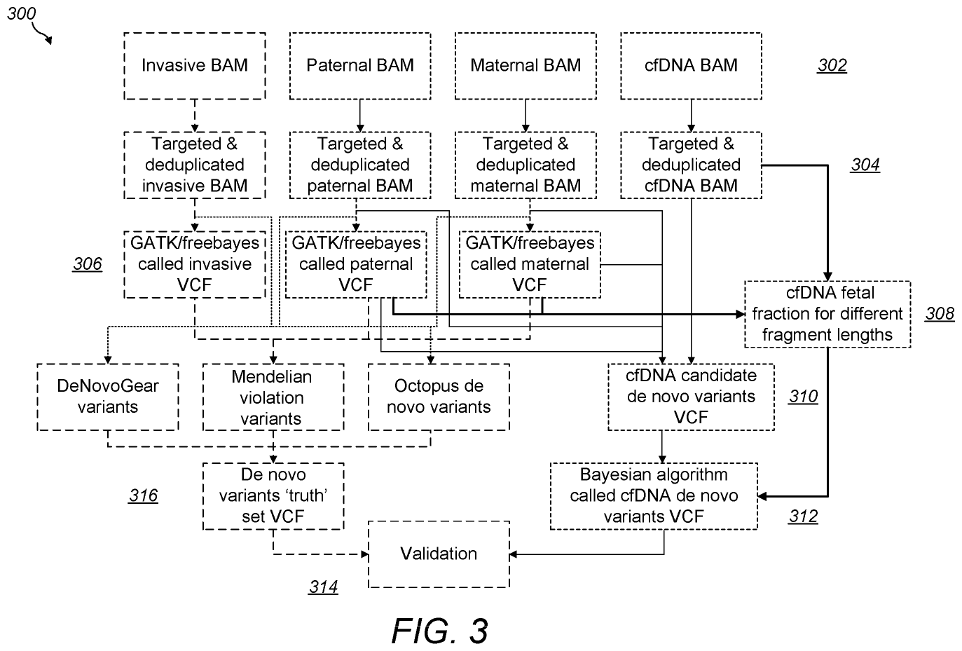

A processing module may be configured to pre-process received samples from the input module by aligning the sequence data to a reference genome before inputting the resultant BAM files 202 (302, FIG. 3) into a purpose built pipeline solution for carrying out the required statistical variant calling steps using the Bayesian framework. This process may be validated 214 (314, FIG. 3).

For validation 214 (314, FIG. 3) and determination of the “truth” sets 216 (316, FIG. 3) for both inherent and de novo variants, various algorithms may be applicable. As shown in the right section of FIGS. 2 and 3, the small dotted lines starting from “targeted” and “deduplicated” invasive and paternal BAM files. The small dotted lines illustrate the input of the BAM files processed by the GATK/Freebayes 206 (306, FIG. 3), and serve as input to software such as DeNovoGear and Octopus (used to analyze de novel mutations) for assessing variants. The dash lines are also shown on the right section of FIGS. 2 and 3 illustrate the input of various Variant Call Format (VCF) files into the Mendelian violation step (i.e. from bcftools or other tools for manipulating variant calls in the VCF and/or its binary counterpart BCF) and the inherited variants truth set. Accordingly, the de novo variants “truth” set is created from the intersection of the results from the three preceding steps shown in the figure (DeNovoGear+Octopus+Mendelian violation). The inherited variants “truth” set is the intersection of variants called in the invasive sample VCF, and variants, which are called with respect to at least the de novo variants “truth” set VCF.

The dark solid lines are shown on the left side of FIGS. 2 and 3. The dark solid lines illustrate further processing of the BAM files 202 (302, FIG. 3). For example, further processing may entail the removal of duplicated reads, for example, flagging the duplicated reads and providing a new set of reads without the flagged duplicates. The duplicate reads may be discounted in a targeted manner, thus making them less relevant. The targeted and duplicated resultant BAM files 204 (304, FIG. 3) may in turn be further processed using variant caller or detector applying software such as GATK/Freebayes 206 (306, FIG. 3) to produce and generate the VCFs from the BAM files as part of the statistical variant calling steps.

Following the variant calling, a Bayesian framework is applied to compute the probability of fetal genotypes based on the samples. The probability of tested potential fetal genotypes may be computed or calculated using the Bayesian framework with respect to the presence of potential fetal genotypes within a fetal-derived fraction of the samples.

The Bayesian framework is adapted to calculate a posterior probability of whether a DNA sequence variant is present in the samples based on a prior probability and a likelihood function that computes the P (genetic sequence data|fetal genotypes), as described in sections above. The prior probability in turn may be calculated based on an expected mutation rate for a de novo DNA sequence variant, or the probability of Mendelian inheritance for an inherited DNA sequence variant. Here, in the figures, the light solid lines represent the inputs between the various modules with respect to the Bayesian framework. For example, the resultant VCF of cfDNA candidate variants 210 (310, FIG. 3) may serve as input to the Bayesian framework for computing or detecting the presence of cfDNA candidate variants or Bayesian algorithm called cfDNA candidate variants 212 (312, FIG. 3) described therein. The Bayesian framework may receive inputs of VCFs generated from variant callers with respect to both parents.

In addition, the different fragment lengths of fetal-derived DNA in the genomic sequence data and DNA from the parents may be used to determine the proportion of fetal-derived DNA fraction 208 (308, FIG. 3) in the genomic sequence data. The probability computed using the Bayesian framework may be calculated with respect to the fetal-derived DNA fraction 208 (308, FIG. 3) of the samples.

The system further comprises an output module configured to determine, based on the posterior probability of fetal genotypes, whether the DNA sequence variant is present in a fetus. This determination is effectively based on or considered with respect to the Bayesian algorithm called cfDNA candidate variants 212 (312, FIG. 3). These variants may be validated 214 (312, FIG. 3) using inherited and de novo variants' “truth” sets 216 (316, FIG. 3) for gaging the presence of a DNA sequence variant in the fetus, and whether it is probable that the detected variant using the Bayesian algorithm is a false-positive or a false-negative. The posterior probability of the Bayesian algorithm may thus be recalibrated. One or more machine learning models, trained using data associated with de novo and inherited DNA sequence variants, may be used with respect to the recalibration.

The system may be configured to provide a set of potential DNA sequence variants based on the probability of fetal genotypes. The set of potential DNA sequence variants may comprise variants that are potentially disease-causing or pathogenic DNA sequence variants in the fetus during pregnancy. The set of potential DNA sequence variants may in turn be applied clinically to validate or purposed for guiding treatment or diagnostic decisions made by a user of the system. The system may be adapted to perform one or more methods in relation to the method or system described according to the other figures.

FIG. 3 is a schematic diagram illustrating an example of the Bayesian framework applied to the genomic sequences data for detection of de novo DNA sequence variants only without having consideration for the inherent. Respective lines show inputs to respective modules. In relation to FIG. 2, a validated “truth” set is derived from the BAM file obtained from invasive means. However, the called maternal and parental VCFs do not directly serve as input to the Bayesian framework. Instead, the maternal and parental variants that are detected may serve as input to cfDNA candidate de novo variant calculation in addition to fetal-derived DNA fraction calculation for different fragment lengths. As such, the resultant de novo variant predictions are validated against the de novo “truth” set.

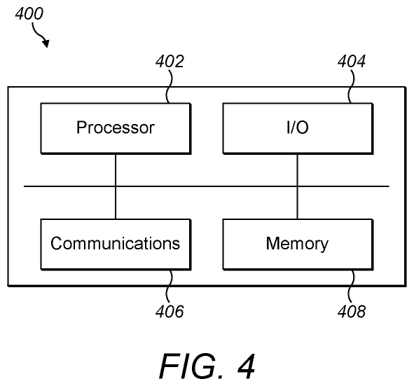

FIG. 4 is a block diagram of a computing device or apparatus suitable for implementing embodiments of the invention. The computing device or apparatus may be used to implement one or more aspects of the present system(s), method(s), and/or process(es) combinations thereof, modifications thereof, and/or as described with reference to FIGS. 1 to 3 and/or as described herein. Computing apparatus/system 400 includes one or more processor unit(s) 402, an input/output unit 404, communications unit/interface 406, a memory unit 408 in which the one or more processor unit(s) 402 are connected to the input/output unit 404, communications unit/interface 406, and the memory unit 408. In some embodiments, the computing apparatus/system 400 may be a server, or one or more servers networked together. In some embodiments, the computing apparatus/system 400 may be a computer or supercomputer/processing facility or hardware/software suitable for processing or performing the one or more aspects of the present system(s), apparatus, method(s), and/or process(es) combinations thereof, modifications thereof, and/or as described with reference to FIGS. 1 to 3 and/or as described herein. The communications interface 406 may connect the computing apparatus/system 400, via a communication network, with one or more services, devices, the server system(s), cloud-based platforms, systems for implementing subject-matter databases and/or knowledge graphs for implementing the invention as described herein. The memory unit 408 may store one or more program instructions, code or components such as, by way of example only but not limited to, an operating system and/or code/component(s) associated with the process(es)/method(s) as described with reference to FIGS. 1 to 3, additional data, applications, application firmware/software and/or further program instructions, code and/or components associated with implementing the functionality and/or one or more function(s) or functionality associated with one or more of the method(s) and/or process(es) of the device, service and/or server(s) hosting the present process(es)/method(s)/system(s), apparatus, mechanisms and/or system(s)/platforms/architectures for implementing the invention as described herein, combinations thereof, modifications thereof, and/or as described with reference to at least one of the FIGS. 1 to 3.

In the embodiments, examples, and aspects of the invention as described herein, such as process(es), method(s), system(s) may be implemented on and/or comprise one or more cloud platforms, one or more server(s) or computing system(s) or device(s). A server may comprise a single server or network of servers; the cloud platform may include a plurality of servers or network of servers. In some examples, the functionality of the server and/or cloud platform may be provided by a network of servers distributed across a geographical area, such as a worldwide distributed network of servers, and a user may be connected to an appropriate one of the network of servers based upon a user location and the like. The following describes further aspects of the present disclosure in relation to one or more embodiments.

In one aspect is a method for determining DNA sequence variation in a fetus from samples of the fetus and parents using a Bayesian framework, the method comprising: receiving the samples comprise genomic sequence data covering one or more genomic regions of interest in relation to the fetus and the parents; processing the received samples at least in part based on a reference genome; computing a probability of fetal genotypes based on the samples using the Bayesian framework, wherein the Bayesian framework computes a posterior probability of whether a DNA sequence variant is present in the samples based on a prior probability and a likelihood function; and determining, based on the posterior probability of fetal genotypes, whether the DNA sequence variant is present in the fetus.

In another aspect is a system for DNA sequence variant detection using a Bayesian framework, wherein the system comprising: an input module configured to receive samples comprise genomic sequence data covering one or more genomic regions of interest in relation to at least one parent; a processing module configured to pre-process received samples at least in part based on a reference genome; a Bayesian framework configured to compute a probability of fetal genotypes based on the samples, wherein the Bayesian framework is adapted to calculate a posterior probability of whether a DNA sequence variant is present in the samples based on a prior probability and a likelihood function; and an output module configured to determine, based on the posterior probability of fetal genotypes, whether the DNA sequence variant is present in a fetus.

In another aspect is a system for DNA sequence variant detection using a Bayesian framework, wherein the system comprising: an input module configured to receive samples comprise genomic sequence data covering one or more genomic regions of interest in relation to the fetus and the parents; a processing module configured to pre-process received samples at least in part based on a reference genome; a Bayesian framework configured to compute a probability of fetal genotypes based on the samples, wherein the Bayesian framework is adapted to calculate a posterior probability of whether a DNA sequence variant is present in the samples based on a prior probability and a likelihood function; and an output module configured to determine, based on the posterior probability of fetal genotypes, whether the DNA sequence variant is present in a fetus.

In another aspect is a system for DNA sequence variant detection using a Bayesian framework, wherein the system comprises at least one processor configured to execute method of the first aspect.

In another aspect is a device, the device comprises one or more processors; one or more memory; and one or more programs, wherein said one or more programs are stored in said one or more memory and configured to be executed by said one or more processors; said one or more programs including instructions according to the method the first aspect.

In another aspect is a non-transitory computer readable storage medium storing one or more programs, said one or more programs comprising instructions, which when executed by one or more processors of an electronic device, cause the device to execute the method according the first aspect.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

As an option, further comprising: computing a proportional distribution for the samples representative of a fetal-derived fraction and a maternal-derived fraction of the samples, wherein the proportional distribution is computed based on lengths of DNA fragments associated with each fraction in relation to the genomic sequence data; and applying the proportional distribution in accordance with the Bayesian framework for determining whether the DNA sequence variant is present in the fetus.

As an option, wherein the computing of a probability of fetal genotypes is based on the samples using a Bayesian framework further comprising: calculating the probability of tested potential fetal genotypes being present within a fetal-derived fraction of the samples.

As an option, wherein the prior probability is calculated based on an expected mutation rate for a de novo DNA sequence variant, or the probability of Mendelian inheritance for an inherited DNA sequence variant.

As an option, further comprising: determining, in relation to the Bayesian framework, whether a DNA sequence variant is a de novo DNA sequence variant or an inherited DNA sequence variant for calculating the prior probability.

As an option, further comprising: providing a variable error rate in relation to the likelihood function, wherein the error rate is applied with respect to likelihood calculations of a de novo DNA sequence variant, an inherited DNA sequence variant, and/or a range of inherited alleles based on parental genotypes. As an option, wherein the error rate is provided by an error model. As an option, wherein the error model is configured to use locus-specific error rates based on the genomic sequence data in the likelihood calculations, wherein the likelihood calculations compute P (genetic sequence data|fetal genotypes).

As an option, further comprising: recalibrating the probability of fetal genotypes using one or more machine learning (ML) models to determine whether the DNA sequence variant is a false-positive DNA sequence variant or a false-negative DNA sequence variant. As an option, wherein said one or more ML models are trained using data associated with de novo and inherited DNA sequence variants.

As an option, wherein the samples comprise DNA samples taken from a pregnant mother of the fetus and fetal cfDNA (cffDNA) samples taken from maternal blood of the pregnant mother.

As an option, wherein the samples further comprise DNA samples taken from a father of the fetus.

As an option, wherein the processing the received samples in part based on a reference genome further comprising: aligning the genomic sequence data to the reference genome; and detecting one or more potential variants in the samples based on the aligned genomic sequence data.

As an option, wherein the DNA sequence variants comprise inherited and de novo variants.

As an option, wherein the inherited and de novo variants comprise single-nucleotide polymorphisms (SNVs) and short indels.

As an option, wherein said one or more genomic regions of interest comprise a subset of genes with at least one actionable intervention. As an option, wherein the system is configured to determine DNA sequence variant in the fetus from the samples of the fetus and both parents according to any one of the above options.

As an option, wherein the system is configured to provide a set of potential DNA sequence variants based on the probability of fetal genotypes, wherein the set of potential DNA sequence variants comprises pathogenic DNA sequence variants in the fetus during pregnancy.

As an option, wherein the set of DNA sequence variants is applied clinically to provide actionable interventions, validate or guide treatment or diagnostic decisions made by a user of the system.

It is understood that any of the aspects of present disclosure may be combined or one or more options herein described.

The above description discusses embodiments of the invention with reference to a single user for clarity. It will be understood that in practice the system may be shared by a plurality of users, and possibly by a very large number of users simultaneously.

The embodiments described above may be configured to be semi-automatic and/or are configured to be fully automatic. In some examples a user or operator of the querying system(s)/process(es)/method(s) may manually instruct some steps of the process(es)/method(es) to be carried out.

The described embodiments of the invention a system, process(es), method(s) and the like according to the invention and/or as herein described may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the process/method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium or non-transitory computer-readable medium. Computer-readable media may include, for example, computer-readable storage media. Computer-readable storage media may include volatile or non-volatile, removable or non-removable media implemented in any method or technology for storage of information such as computer-readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection or coupling, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realize that such processing capabilities are incorporated into many different devices and therefore the term ‘computer’ includes PCs, servers, IoT devices, mobile telephones, personal digital assistants and many other devices.

Those skilled in the art will realize that storage devices utilized to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realize that by utilizing conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Aspects should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary”, “example” or “embodiment” is intended to mean “serving as an illustration or example of something”. Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art.

What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A method for determining DNA sequence variation in a fetus from samples of the fetus and parents using a Bayesian framework, the method comprising:
receiving the samples comprise genomic sequence data covering one or more genomic regions of interest in relation to the fetus and the parents;
processing the received samples at least in part based on a reference genome;
computing a probability of fetal genotypes based on the samples using the Bayesian framework, wherein the Bayesian framework computes a posterior probability of whether a DNA sequence variant is present in the samples based on a prior probability and a likelihood function; and
determining, based on the posterior probability of fetal genotypes, whether the DNA sequence variant is present in the fetus.

2. The method of claim 1, further comprising:
computing a proportional distribution for the samples representative of a fetal-derived fraction and a maternal-derived fraction of the samples, wherein the proportional distribution is computed based on lengths of DNA fragments associated with each fraction in relation to the genomic sequence data; and
applying the proportional distribution in accordance with the Bayesian framework for determining whether the DNA sequence variant is present in the fetus.

3. The method of claim 1, wherein said computing a probability of fetal genotypes based on the samples using a Bayesian framework, further comprising:
calculating the probability of tested potential fetal genotypes being present within a fetal-derived fraction of the samples.

4. The method of claim 1, wherein the prior probability is calculated based on an expected mutation rate for a de novo DNA sequence variant, or the probability of Mendelian inheritance for an inherited DNA sequence variant.

5. The method of claim 1, further comprising:
determining, in relation to the Bayesian framework, whether a DNA sequence variant is a de novo DNA sequence variant or an inherited DNA sequence variant for calculating the prior probability.

6. The method of claim 1, further comprising:
providing a variable error rate in relation to the likelihood function, wherein the error rate is applied with respect to likelihood calculations of a de novo DNA sequence variant, an inherited DNA sequence variant, and/or a range of inherited alleles based on parental genotypes.

7. The method of claim 6, wherein the error rate is provided by an error model.

8. The method of claim 7, wherein the error model is configured to use locus-specific error rates based on the genomic sequence data in the likelihood calculations, wherein the likelihood calculations compute P (genetic sequence data|fetal genotypes).

9. The method of claim 1, further comprising:
recalibrating the probability of fetal genotypes using one or more machine learning (ML) models to determine whether the DNA sequence variant is a false-positive DNA sequence variant or a false-negative DNA sequence variant.

10. The method of claim 9, wherein said one or more ML models are trained using data associated with de novo and inherited DNA sequence variants.

11. The method of claim 1, wherein the samples comprise DNA samples taken from a pregnant mother of the fetus and fetal cfDNA (cffDNA) samples taken from maternal blood of the pregnant mother.

12. The method of claim 1, wherein the samples further comprise DNA samples taken from a father of the fetus.

13. The method of claim 1, wherein the processing the received samples in part based on a reference genome further comprising:
aligning the genomic sequence data to the reference genome; and
detecting one or more potential variants in the samples based on the aligned genomic sequence data.

14. The method of claim 1, wherein the DNA sequence variants comprise inherited and de novo variants.

15. The method of claim 14, wherein the inherited and de novo variants comprise single-nucleotide polymorphisms (SNVs) and short indels.

16. The method of claim 1, wherein said one or more genomic regions of interest comprise a subset of genes with at least one actionable intervention.

17. A system for DNA sequence variant detection using a Bayesian framework, wherein the system comprising:
an input module configured to receive samples comprise genomic sequence data covering one or more genomic regions of interest in relation to at least one parent;
a processing module configured to pre-process received samples at least in part based on a reference genome;
a Bayesian framework configured to compute a probability of fetal genotypes based on the samples, wherein the Bayesian framework is adapted to calculate a posterior probability of whether a DNA sequence variant is present in the samples based on a prior probability and a likelihood function; and
an output module configured to determine, based on the posterior probability of fetal genotypes, whether the DNA sequence variant is present in a fetus.

18. The system of claim 17, wherein the system configured to determine DNA sequence variant in the fetus from the samples of the fetus and both parents according to a method for determining DNA sequence variation in a fetus from samples of the fetus and parents using a Bayesian framework, the method comprising:
receiving the samples comprise genomic sequence data covering one or more genomic regions of interest in relation to the fetus and the parents;
processing the received samples at least in part based on a reference genome;
computing a probability of fetal genotypes based on the samples using the Bayesian framework, wherein the Bayesian framework computes a posterior probability of whether a DNA sequence variant is present in the samples based on a prior probability and a likelihood function; and
determining, based on the posterior probability of fetal genotypes, whether the DNA sequence variant is present in the fetus,
wherein the method further comprises computing a proportional distribution for the samples representative of a fetal-derived fraction and a maternal-derived fraction of the samples, wherein the proportional distribution is computed based on lengths of DNA fragments associated with each fraction in relation to the genomic sequence data; and
applying the proportional distribution in accordance with the Bayesian framework for determining whether the DNA sequence variant is present in the fetus.

19. The system of claim 17, wherein the system is configured to provide a set of potential DNA sequence variants based on the probability of fetal genotypes, wherein the set of potential DNA sequence variants comprises pathogenic DNA sequence variants in the fetus during pregnancy.

20. The system of claim 17, wherein the set of DNA sequence variants is applied clinically to provide actionable interventions, validate or guide treatment or diagnostic decisions made by a user of the system.
